(19) United States
(12) Patent Application Publication
(10) Pub. No.: US 2016/0113820 A1
(43) Pub. Date: Apr. 28, 2016

(54) SHOES FOR CAST-WORN PATIENT

(71) Applicant: JAE MIN HAN, Seoul (KR)

(72) Inventor: JAE MIN HAN, Seoul (KR)

(21) Appl. No.: 14/819,410

(22) Filed: Aug. 5, 2015

(30) Foreign Application Priority Data
Oct. 27, 2014 (KR) 20-2014-0007787

(51) Int. Cl. A61F 13/04

(52) U.S. Cl. CPC A61F 13/043

(57) ABSTRACT

According to an embodiment of the present disclosure, a shoe for a patient with a cast in a foot comprises a bottom plate supporting the foot, a cover piece covering the foot and having a first attaching means at a portion corresponding to an instep of the foot, and a protection piece extending from a front portion of the bottom plate where toes of the foot are positioned, bent to cover the toes, and having a second attaching means at a position corresponding to the first attaching means, the second attaching means attached to the first attaching means, wherein the bottom plate, the cover piece, and the protection piece are formed as a single body.

### CROSS-REFERENCE TO RELATED APPLICATIONS

This patent application claims priority under 35 U.S.C. §119 to Korean Utility Model Application No. 20-2014-0007787, filed on Oct. 27, 2014, in the Korean Intellectual Property Office, the disclosure of which is incorporated by reference herein in its entirety.

### TECHNICAL FIELD

The present disclosure relates to a shoe for a cast-worn patient, and more specifically, to a shoe for a patient with his foot in a cast, which may protect the patient's toes while wearing the shoe.

### DISCUSSION OF RELATED ART

A plaster cast (shortly, a “cast”) is a shell, frequently made from plaster, encasing an injured foot to stabilize and hold anatomical structures, most often a broken bone (or bones), in place until healing is confirmed. A patient with his foot in a cast has difficulty wearing a shoe. There are a few types of conventional shoes that apply to the patient wearing a cast in his foot. However, such conventional shoes generally leave the patient's toes exposed to the outside. Accordingly, a need exists for a shoe for a patient that may protect the toes while fitting the cast-worn foot.

### SUMMARY

According to an embodiment of the present invention, a shoe for a patient with a cast in a foot comprises a bottom plate supporting the foot, a cover piece covering the foot and having a first attaching means at a portion corresponding to an instep of the foot, and a protection piece extending from a front portion of the bottom plate where toes of the foot are positioned, bent to cover the toes, and having a second attaching means at a position corresponding to the first attaching means, the second attaching means attached to the first attaching means, wherein the bottom plate, the cover piece, and the protection piece are formed as a single body.

The bottom plate, the cover piece, and the protection piece may be formed of ethylene vinyl acetate (EVA).

The bottom plate has a thickness of 2 cm to 3 cm, and the cover piece and the protection piece each may have a thickness of 1 mm to 5 mm.

The first attaching means may be formed on each of both parts of the cover piece covering the instep of the foot from both sides of the bottom plate. The second attaching means may be formed on an overall portion of the protection piece that meets the first attaching means. The first attaching means and the second attaching means may be formed of Velcro tapes.

### BRIEF DESCRIPTION OF THE DRAWINGS

A more complete appreciation of the present disclosure and many of the attendant aspects thereof will be readily obtained as the same becomes better understood by reference to the following detailed description when considered in connection with the accompanying drawings, wherein:

FIG. 1 is a perspective view illustrating a shoe for a cast-worn patient before a protection piece is coupled according to an embodiment of the present disclosure;

FIG. 2 is a cross-sectional view illustrating a shoe for a cast-worn patient according to an embodiment of the present disclosure;

FIG. 3 is a perspective view illustrating a shoe for a cast-worn patient after a protection piece is coupled according to an embodiment of the present disclosure;

FIG. 4 is a cross-sectional view illustrating a shoe for a cast-worn patient according to an embodiment of the present disclosure;

FIG. 5 is a perspective view illustrating a shoe for a cast-worn patient in which a cover piece and a protection piece are open according to an embodiment of the present disclosure;

FIG. 6 is a perspective view illustrating a shoe for a cast-worn patient in which a protection piece is bent according to an embodiment of the present disclosure; and

FIGS. 7 and 8 are views illustrating examples of using a shoe for a cast-worn patient according to embodiments of the present disclosure.

### DETAILED DESCRIPTION OF EXEMPLARY EMBODIMENTS

Hereinafter, exemplary embodiments of the inventive concept will be described in detail with reference to the accompanying drawings. The same reference denotations may be used to refer to the same or similar elements throughout the specification and the drawings. The inventive concept, however, may be modified in various different ways, and should not be construed as limited to the embodiments set forth herein. As used herein, the singular forms “a,” “an,” and “the” are intended to include the plural forms as well, unless the context clearly indicates otherwise. It will be understood that when an element or layer is referred to as being “on,” “connected to,” “coupled to,” or “adjacent to” another element or layer, it can be directly on, connected, coupled, or adjacent to the other element or layer, or intervening elements or layers may be present.

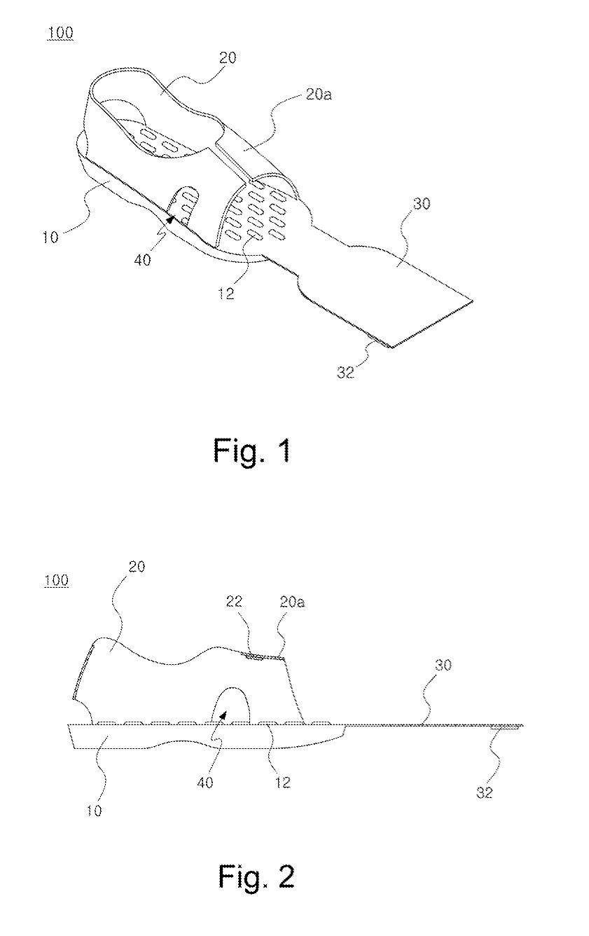

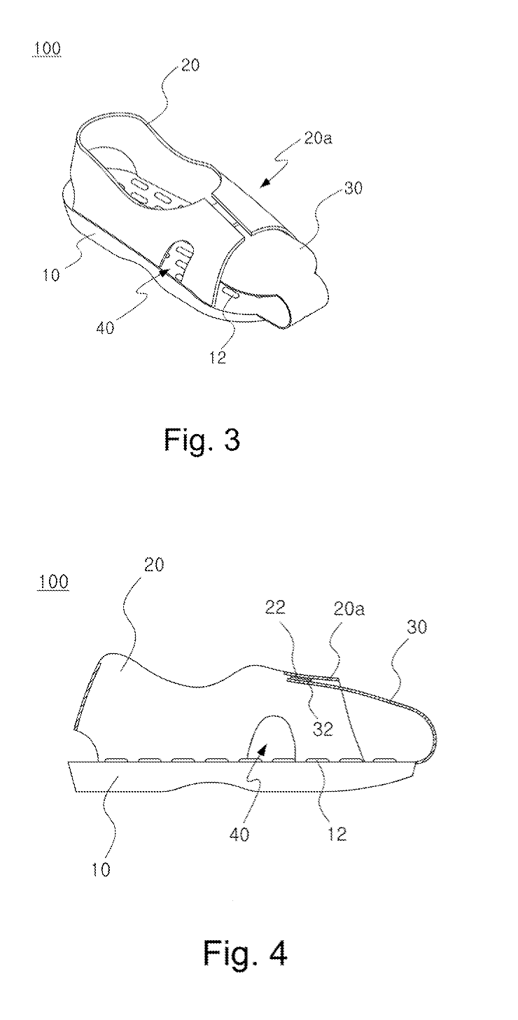

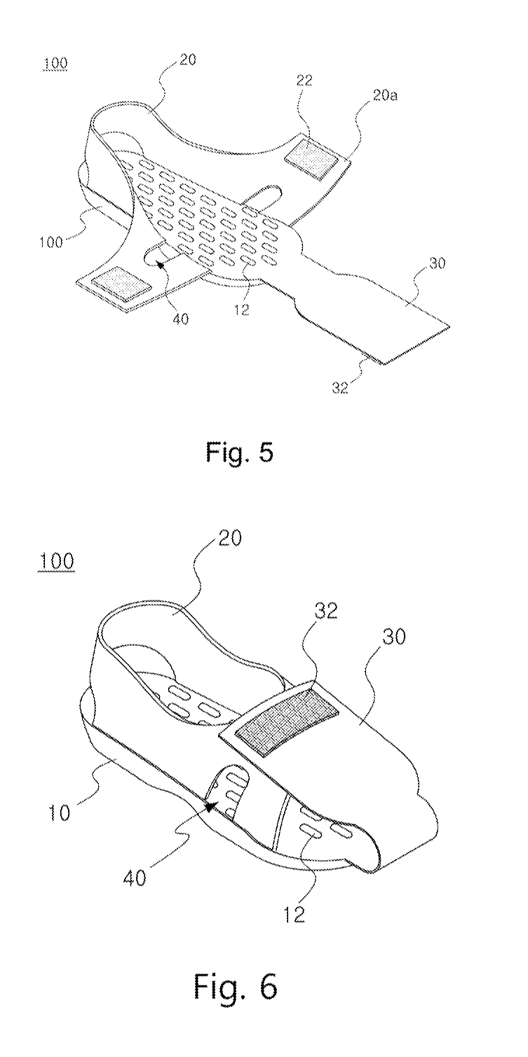

FIG. 1 is a perspective view illustrating a shoe for a cast-worn patient before a protection piece is coupled according to an embodiment of the present disclosure. FIG. 2 is a cross-sectional view illustrating a shoe for a cast-worn patient according to an embodiment of the present disclosure. FIG. 3 is a perspective view illustrating a shoe for a cast-worn patient after a protection piece is coupled according to an embodiment of the present disclosure. FIG. 4 is a cross-sectional view illustrating a shoe for a cast-worn patient according to an embodiment of the present disclosure. FIG. 5 is a perspective view illustrating a shoe for a cast-worn patient in which a cover piece and a protection piece are open according to an embodiment of the present disclosure. FIG. 6 is a perspective view illustrating a shoe for a cast-worn patient in which a protection piece is bent according to an embodiment of the present disclosure.

Referring to FIGS. 1 to 6, the shoe 100 for a cast-worn patient (hereinafter, simply referred to as a shoe 100) may be used by a patient who wears a cast on his foot except for the toes when the patient is out. The shoe 100 may include a bottom plate 10, a cover piece 20, and a protection piece 30. The shoe 100 may support the patient's foot while allowing him to walk safely.

The bottom plate 10 may be prepared as a plate to have a shape corresponding to the shape of the patient's cast-worn foot. The bottom plate 10 may prevent the patient's foot from directly contacting the ground. The bottom plate 10 may have a predetermined cushion to distribute the patient's weight to minimize the influence on the injured part.

The bottom plate 10 may have a plurality of protrusions on the surface that contacts the patient's foot. The protrusions may prevent slippery.

The bottom plate 10 may be formed of, e.g., ethylene vinyl acetate (EVA) that is highly elastic, soft, and easy-to-treat, and the bottom plate 10 may have a thickness of about 2 cm to 3 cm to provide a predetermined cushion.

Although not clearly shown, the bottom surface of the bottom plate 10 may have various shapes or structures considering friction against the ground.

The cover piece 20 is formed on the bottom plate 10. The cover piece 20 may be shaped to cover the overall injured part of the patient, with the injured part therein.

The cover piece 20 may have a rear part at least partially covering the heel and an open front part that exposes the toes. The cover piece 20 may include attaching parts 20a that are respectively placed on both sides of the instep and a first attaching means 22 under the attaching parts 20a to contact the instep.

The first attaching means 22 may include one of a Velcro tape with a coarse surface or a Velcro tape with a soft surface.

For example, the first attaching means 22 may include a Velcro tape with a soft surface to prevent the patient's injured part or cast from being negatively affected.

The cover piece 20 may have at least one or more vents 40 at both sides and a rear side thereof, enabling sweat to easily pass out therethrough while giving a good appearance. The shape of the vents 40 is not limited to those shown in the drawings. For example, each vent 40 may have various shapes, such as a heart or star. Further, the vents 40 may be formed at various positions.

The cover piece 20 may be integrally formed with of the EVA bottom plate 10. For example, the cover piece 20 may also be formed of EVA. The cover piece 20 may have a thickness of 1 mm to 5 mm.

The protection piece 30 may be provided to protect the patient's toes exposed from the cast. The protection piece 30 may extend from a front part of the bottom plate 10. For example, the protection piece 30 may be bent to receive the exposed toes while the patient places his cast-worn foot on the bottom plate 10. In this state, the protection piece 30 may be attached to the first attaching means 22. The protection piece 30 may include a second attaching means 32 that may be attached to the first attaching means 22. The second attaching means 32 may be formed on a bottom surface of an end portion of the protection piece 30.

The second attaching means 32 may be formed on an overall portion of the protection piece where the first attaching means 22 may meet, enabling application to patients with various foot lengths.

The second attaching means 32 may include one of a Velcro tape with a coarse surface or a Velcro tape with a soft tape. For example, when the first attaching means 22 includes a Velcro tape with a soft surface, the second attaching means 32 may include a Velcro tape with a coarse surface, and vice versa.

The protection piece 30 may be formed of EVA, and the protection piece 30, together with the bottom plate 10 and the cover piece 20, may be formed as a single body. The protection piece may be bent to cover the patient's toes and to be attached to the first attaching means 22. The protection piece 30 may have a thickness of 1 mm to 5 mm, like the cover piece 20, to stably protect the patient's toes. The protection piece 30 may be formed to cover a front and top surface of the toes but not to cover side surfaces of the toes, enabling sweat or smell to easily pass out.

The shape or structure of the protection piece 30 is not limited thereto, and the protection piece 30 may have other various structures and shapes.

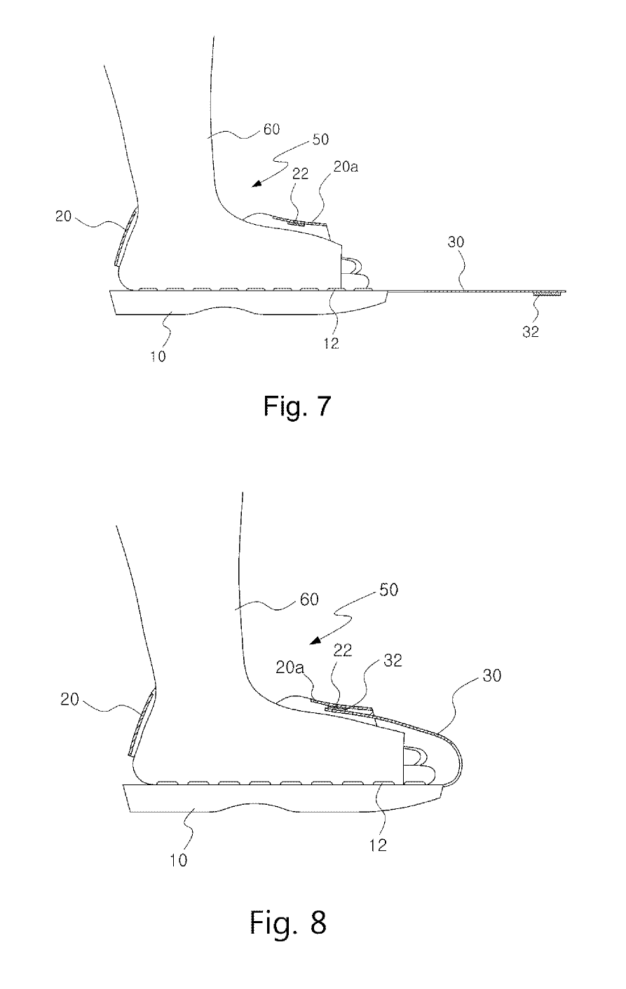

FIGS. 7 and 8 are views illustrating examples of using a shoe for a cast-worn patient according to embodiments of the present disclosure.

Referring to FIG. 7, the patient's foot 50 in a cast 60 is placed in the shoe 100, which includes the bottom plate 10, the cover piece 20, and the protection piece 30, to be covered by the cover piece 20. The bottom plate 10 backs up the cast-worn foot 50, and the cover piece 20 covers the foot 50. The attaching parts 20a are positioned on the instep of the foot 50. The protection piece extends long from the front of the bottom plate 10 without covering the patient's exposed toes.

Referring to FIG. 8, the protection piece 30 is bent to cover the exposed toes and their adjacent instep, and the second attaching means 32 of the protection piece 30 is inserted to be positioned under the attaching parts 20a of the cover piece 20. The second attaching means 32 is attached to the attaching parts 20a of the cover piece 20. The degree by which the protection piece 30 may be adjusted to fit patients with various foot lengths.

As such, the patient with his foot in the cast 60 may wear the shoe 100 while protecting his toes. Accordingly, the toes exposed out of the cast 60 may be protected from any possible harm or injury.

According to an embodiment of the present disclosure, the shoe 100 may be formed of EVA that is elastic, soft, and easy-to-treat. The shoe 100 may be formed by injection molding. The bottom plate, cover piece, and protection piece of the shoe 100 may be simultaneously formed as a single body by injection molding.

Accordingly, the shoe 100 may be easily manufactured with saved costs.

According to an embodiment of the present disclosure, the logo of a hospital or manufacturer may be attached or engraved on the cover piece or protection piece.

While the inventive concept has been shown and described with reference to exemplary embodiments thereof, it will be apparent to those of ordinary skill in the art that various changes in form and detail may be made thereto without departing from the spirit and scope of the inventive concept as defined by the following claims.

What is claimed is:

1. A shoe for a patient with a cast in a foot, the shoe comprising:
a bottom plate supporting the foot;
a cover piece covering the foot and having a first attaching means at a portion corresponding to an instep of the foot; and
a protection piece extending from a front portion of the bottom plate where toes of the foot are positioned, bent to cover the toes, and having a second attaching means at a position corresponding to the first attaching means, the second attaching means attached to the first attaching means, wherein the bottom plate, the cover piece, and the protection piece are formed as a single body.

2. The shoe of claim 1, wherein the bottom plate, the cover piece, and the protection piece are formed of ethylene vinyl acetate (EVA).

3. The shoe of claim 1, wherein the bottom plate has a thickness of 2 cm to 3 cm, and the cover piece and the protection piece each have a thickness of 1 mm to 5 mm.

4. The shoe of claim 1, wherein the first attaching means is formed on each of both parts of the cover piece covering the instep of the foot from both sides of the bottom plate, wherein the second attaching means is formed on an overall portion of the protection piece that meets the first attaching means, and wherein the first attaching means and the second attaching means are formed of Velcro tapes.
